(19) United States
(12) Patent Application Publication
(10) Pub. No.: US 2010/0196475 A1
(43) Pub. Date: Aug. 5, 2010

(54) CONTROLLED RELEASE TABLET FORMULATION CONTAINING MAGNESIUM ALUMINOMETASILICATE

(76) Inventors: Pascal Grenier, Kappelen (FR); Alain Nhamias, Bartenheim (FR); Guy Vergnault, Kembs (FR)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/451,525

(22) PCT Filed: May 16, 2008
(86) PCT No.: PCT/IB2008/002128

§ 371 Date: Mar. 26, 2010

(30) Foreign Application Priority Data
May 17, 2007 (GB) 0709541.7

(51) Int. Cl. A61K 9/28; A61K 9/20; A61K 31/404; A61K 31/554; A61K 31/573; A61P 9/02; A61P 9/06; A61P 25/16; A61P 25/28; A61P 29/00; A61P 11/06

(52) U.S. Cl. 424/474; 424/464; 514/415; 514/211.07; 514/179

(57) ABSTRACT

The present invention relates to a controlled pharmaceutical dosage forms for oral administration, and in particular to the excipients used to prepare such medicaments. For example, a dosage form for oral administration is provided consisting of a minimum of 15% w/w of magnesium aluminometasilicate, one or more pharmaceutically active agents and optionally one or more pharmaceutically acceptable diluents.

The present invention relates to controlled release pharmaceutical dosage forms for oral administration and in particular to the excipients used to prepare such medicaments.

Pharmaceutical dosage forms for oral administration which have controlled release (also referred to as delayed release or sustained release) properties with respect to the release kinetics of the pharmaceutically active agent have proved to be advantageous in overcoming the problems associated with the pharmacology of many drugs which, whilst being suitable for the treatment of a disease condition, have associated toxicological side effects if administered in too great a dose, or require the administration of a large number of tablets to a patient during the course of a day. A controlled release pharmaceutical dosage form is able to provide a sustained release of the active agent from a single tablet over a defined period of time thus avoiding the problems of fast-burst release and/or patient compliance.

The pharmaceutical formulation technology that enabled the development of such controlled release tablets has depended on the use of polymeric substances, for example water swellable and/or gellable polymeric substances, that are initially inert in an aqueous environment but then subsequently swell and/or gel in an aqueous environment (such as the intestine of a patient), thus opening up pores through which the active agent can be released. Examples of such polymers are hydroxypropyl methylcellulose (HPMC) and carboxy methyl cellulose (CMC). There are many other polymer substances used for similar reasons because of their physical/chemical characteristics.

However, the swelling and eroding behaviour of polymers such as HPMC is known to depend on the nature of aqueous environment into which the tablet is placed. The release of the active agent can therefore be dependent on such variables as pH, ionic strength and agitation or other dissolution conditions. The “gel strength” of these polymer components is believed to drive the release of the active agent from the tablet. The tablets or oral dosage forms prepared from such polymers are also vulnerable to the affects of the in vivo environment after administration of the tablet, such as for example the well known “food-effect”.

It has now been surprisingly found that magnesium aluminometasilicate, an excipient previously used in tablet manufacture as a disintegrant, can be used in a different manner to prepare controlled release pharmaceutical dosage forms which overcomes or at least ameliorates these problems and avoids the use of water swellable and/or gellable polymeric substances as the controlled release excipient.

According to a first aspect of the invention, there is provided a dosage form for oral administration consisting of a minimum 15% w/w of magnesium aluminometasilicate, one or more pharmaceutically active agents and optionally one or more pharmaceutically acceptable diluents.

The dosage form may be a tablet of any suitable construction for oral administration to a patient. It may be a multi-layer tablet composition or a single oral dosage form or tablet.

Magnesium aluminometasilicate can be described by the chemical formula Al₂O₃.MgO.2SiO₂.xH₂O and preferably the aluminium oxide is present in the range of from 25% to 40%, the magnesium oxide present in the range of from 10% to 15%, and the silicon dioxide is present in the range of from 25% to 40%. As a substance that absorbs moisture, these percentages are based on drying the substance at 110° C. for 7 hours. In a preferred embodiment of the invention the magnesium aluminometasilicate may be Neusilin™ as produced by Fuji Chemical Industry Co., Ltd. (www.fujichemusa.com).

The controlled-release properties of magnesium aluminometasilicate are exhibited when the proportion of the excipient in the oral dosage form is present at a minimum of 15% w/w. The magnesium aluminometasilicate may be present in the range of from 15% to 95%, suitably of from 40% to 90% or from 45% to 95%, with preferred suitable proportions of 35%, 40%, 45%, 50%, 55%, 60%, 65%, 70%, 75%, 80% 85%, 90% or 95% depending upon the active agent to be released from the oral dosage form (all percentages given as w/w). The controlled-release effect of magnesium aluminometasilicate may also depend on the water solubility of the active substance. So, for a poorly soluble or low-solubility active substance, a lower amount of magnesium aluminometasilicate may be required.

Pharmaceutically acceptable diluents include, but are not limited to, mannose, starch, mannitol, lactose, sorbitol, xylitol, talc, stearic acid, sodium benzoate, magnesium stearate, colloidal silica, maltodextrin, and other excipients known to the expert in the field.

The pharmaceutically active agent present in the oral dosage form may be any suitable agent required to be formulated for controlled release. As used in the present specification, the term pharmaceutically active agent includes pharmaceuticals as well as other substances having a biological effect, such as food supplements (for example vitamins, minerals, glycosaminoglycans, etc.). The magnesium aluminometasilicate present in the oral dosage form is not used as an absorbent for the pharmaceutically active agent. The active agent is therefore preferably provided as a powdered, anhydrous substance prior to compression to form the oral dosage form.

Any pharmaceutically active substance suitable for oral administration in the form of a tablet can be formulated in an oral dosage form (or tablet) of the present invention. An active substance is therefore a pharmaceutical (drug) with a therapeutic use, such substances also include those for administration for non-therapeutic uses, such as diagnosis of for dietary purposes.

Preferably the active substance may be one aimed at the treatment of chronic diseases, for example, drugs acting on the cardiovascular system, anti-arrhythmics, cardiac stimulants, vasodilators, calcium antagonists, anti-hypertensives, for example anti-adrenergic substances of central and peripheral action or substances acting on the arteriolar musculature, analgesic substances, substances acting on the renin-angiotensin system, anti-hypertensives and diuretics in association, anti-Parkinson's Disease agents, diuretics and drugs for the treatment of Alzheimer's disease, anti-histamines and/or anti-asthmatics.

Examples of active substances which may be used in such pharmaceutical forms are: propranolol, atenolol, pindolol, ropinirole, prazosin, ramipril, spirapril; spironolactone, metipranolol, molsidomine, moxonidina, nadolol, nadoxolol, levodopa, metoprolol, timolol.

Analgesic substances include, but are not limited to, steroidal anti-inflammatory drugs, opioid analgesics, and non-steroidal anti-inflammatory drugs (NSAIDs). The analgesic substance may be a non-steroidal anti-inflammatory drug (NSAID), such as acetyl salicylic acid, salicylic acid, indomethacin, ibuprofen, naproxen, naproxen sodium, flubiprofen, indoprofen, ketoprofen, piroxicam, diclofenac, diclofenac sodium, etodolac, ketorolac, or the pharmaceutically acceptable salts and/or derivatives or mixtures thereof.

Other suitable analgesic substances include, but are not limited to opioid analgesics such as alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tramadol, tilidine and pharmaceutically acceptable salts and/or derivatives or mixtures thereof.

Anti-hypertensive drugs may include, diltiazem, trapidil, urapidil, benziodarone, dipiridamole (dipyridamole), lidoflazine, naphthydrofuryl oxalate, perhexeline maleate, oxyfedrine hydrochloride. Anti-histamines and/or anti-asthmatics may include ephedrine, terfenadine, theophylline or chlorpheniramine.

In the tablets of the present patent application, the active substance to be carried may have a very wide solubility interval in water, e.g. between 0.01 mg/L up to 3000 g/L, preferably between 10 mg/L up to 1000 g/L, or between 0.01mg/L up to 100 g/L.

The active substance is preferably contained in a percentage between 0.05% to 70% by weight of the dosage form (or active layer if the dosage form is multi-layer tablet); more preferred ranges of the active substances are 0.05% to 40%, 0.05% to 30%, 0.05% to 10%, 0.05% to 20%, 25%, 30%, 35%, 40%, 45%, 50%, 55%, 60%, 65%, or to 70%.

Oral dosage forms prepared in accordance with the invention can comprise a single homogeneous tablet composed of a single pharmaceutical formulation as described above, or alternatively, the oral dosage form may comprise a plurality of layers to form a multi-layer tablet. In such multi-layer tablets, one or more of the layers may contain an active agent (or may contain different active agents), and one or more of the layers may act as barrier layers or support layers to assist tablet integrity and to further control the rate of release of the active agent(s) from the layers containing active agent formulated in accordance with the present invention.

An alternative tablet construction is a compression coated tablet, in which the active substance is contained within a core which is contained within an outer barrier layer. In some embodiments, the coating may be complete, in other embodiments, the covering may be partial, so for example when the core is of approximately cylindrical form, the partial coating is applied to the lower basal and lateral sides of the core, leaving the upper surface exposed. Such tablet forms may also be composed of multiple layers.

In some embodiments of the invention, it may be preferred that the tablet is compressed to a hardness of at least 80N, suitably in the range of from 85N to 230N, preferably 90N, to 210N. The controlled release profile of such oral dosage forms can be modulated by increasing the compression pressure where increased pressure leads to increased hardness values which provide slower release of the active over a longer time period.

According to a second aspect of the invention, there is provided a dosage form for oral administration consisting of a minimum 15% w/w of magnesium aluminometasilicate, a pharmaceutically active agent, a pharmaceutically acceptable lipid excipient and optionally one or more pharmaceutically acceptable diluents.

Particularly useful lipid excipients (or waxy or lipoid excipients) for modifying the controlled-release characteristics of magnesium aluminometasilicate include microcrystalline cellulose, which is a form of partially depolymerised alpha cellulose derived from purified wood pulp and available under the general product name of Avicel™ PH, suitably grades PH101 or PH102. Another useful excipient is glyceryl behenate (or tribehenin), suitably in the form of atomised glyceryl behenate formed by esterification of glycerol by behenic acid followed by spray-cooling and available under the product name of Compritol™ 888 ATO.

According to a third aspect of the invention, there is provided a method for controlling the release of a pharmaceutically active agent from a dosage form, the method comprising the step of formulating the active agent in a granulate composition comprising a minimum 15% w/w magnesium aluminometasilicate.

According to a fourth aspect of the invention, there is provided the use of magnesium aluminometasilicate as a controlled-release excipient in the formulation of a pharmaceutically active substance in a dosage form. Aluminometasilicate is used without polymeric materials commonly used in controlled release dosage forms.

Preferred features for the second and subsequent aspects of the invention are as for the first aspect mutatis mutandis.

Generally preferred embodiments of the invention are therefore oral dosage forms consisting of magnesium aluminometasilicate and an active substance, without further components being present. Where the amount of magnesium aluminometasilicate needs to be reduced to take account of the solubility of the active substance, the remainder of the tablet can be prepared from a pharmaceutically acceptable diluent, such as lactose or mannose. Other preferred embodiments of the invention are oral dosage forms consisting of magnesium aluminometasilicate, an active substance and a pharmaceutically acceptable lipid excipient, such as microcrystalline cellulose and/or glyceryl behenate.

The invention will now be further described by way of reference to the following Examples and Figures which are provided for the purposes of illustration only and are not to be construed as being limiting on the invention. Reference is made to a number of Figures in which:

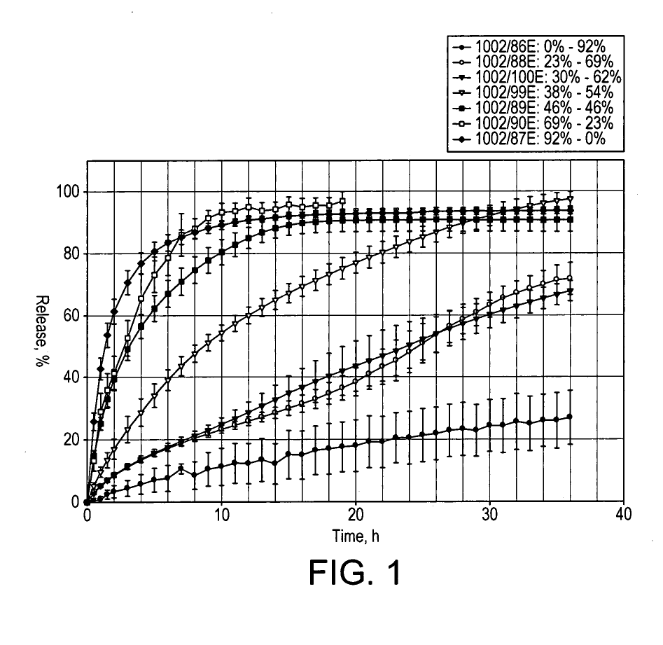

FIG. 1 shows the dissolution profiles for tablets 86E, 88E, 100E, 99E, 89E, 90E and 87E where the Neusilin™ content has been decreased from 92% w/w to 0% w/w.

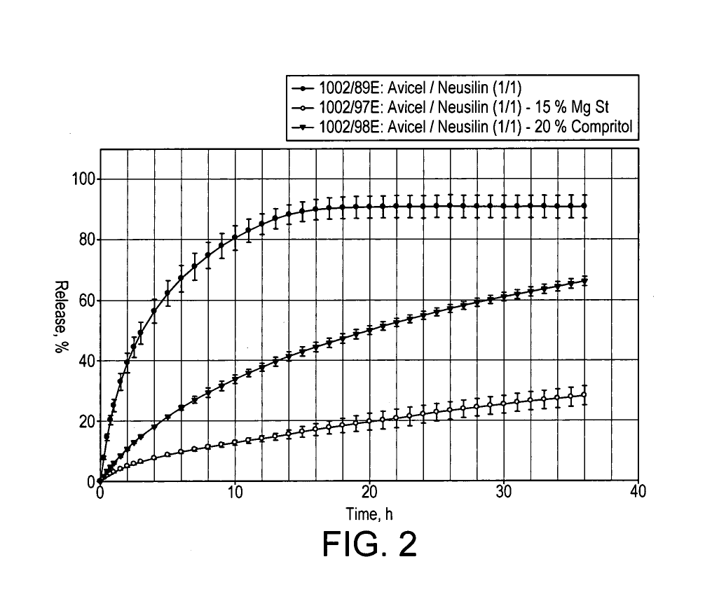

FIG. 2 shows the dissolution profiles for tablets 89E (no Compritol™ 888 ATO), 98E (19.2% w/w Compritol™888 ATO) and 97E (14.9% w/w magnesium stearate).

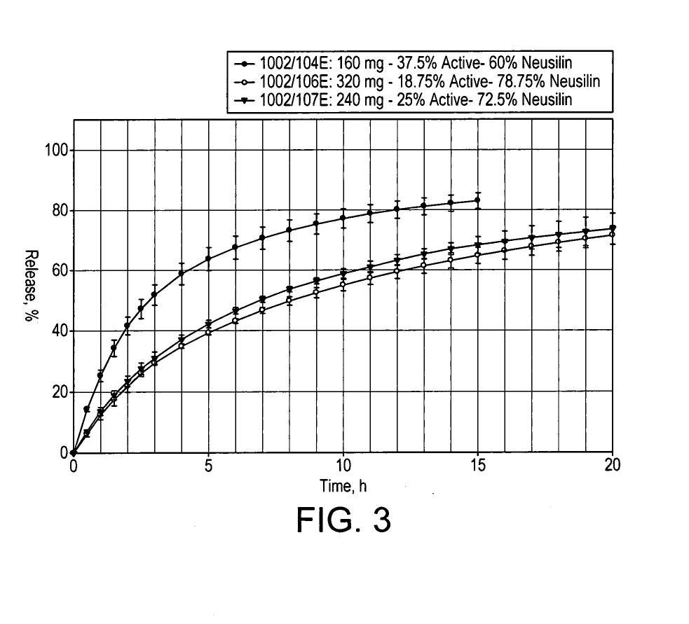

FIG. 3 shows the dissolution profiles for tablets 104E (37.5% w/w active and 60% w/w Neusilin™), 107E (25% w/w active and 72.5% w/w Neusilin™) and 106E (18.75% w/w active and 78.75% w/w Neusilin™).

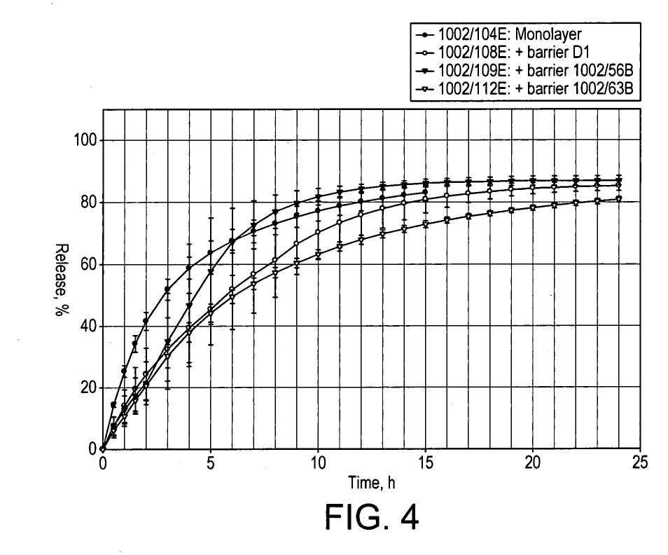

FIG. 4 shows the dissolution profiles for tablets 104E, 108E (104E+D1), 109E (104E+56B) and 112E (104E+63B).

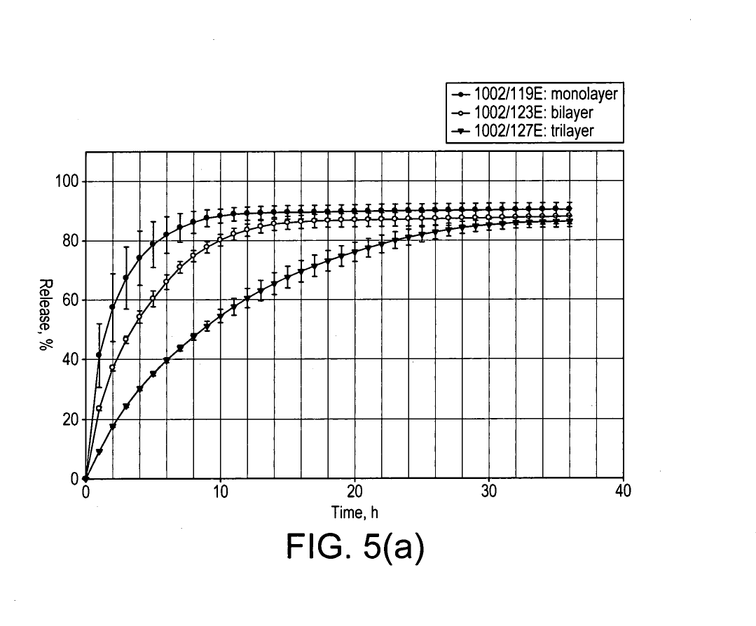

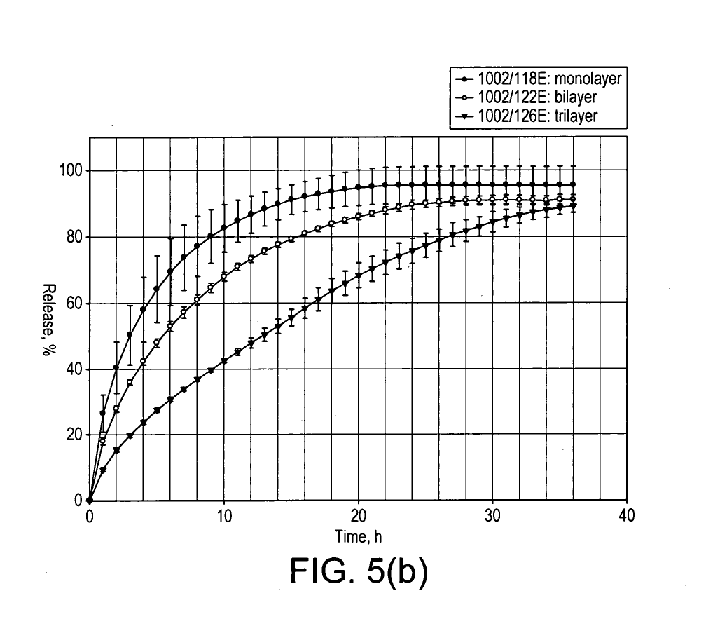

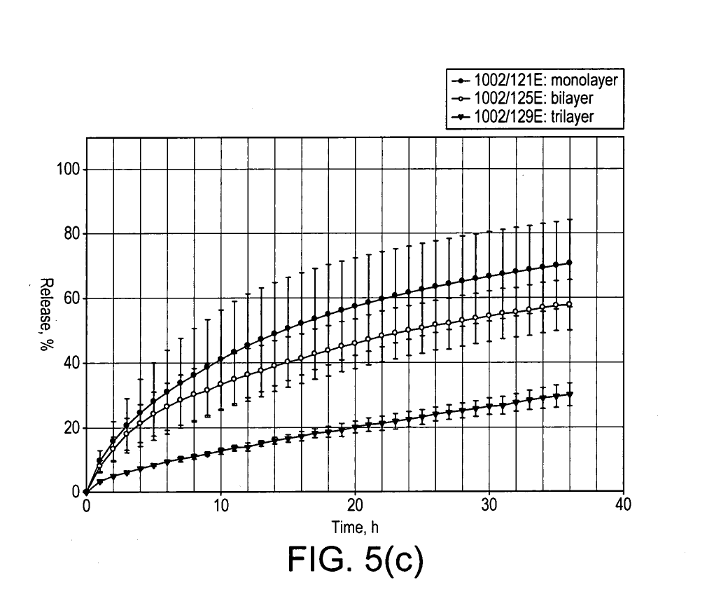

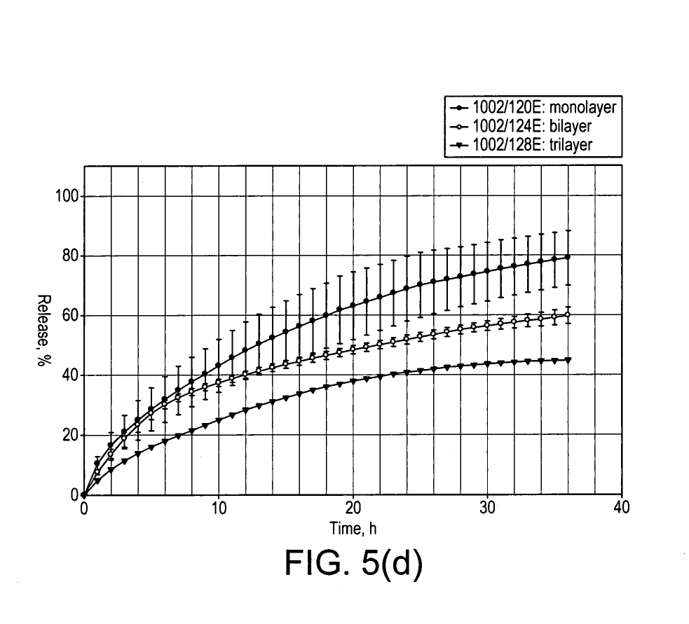

FIG. 5 shows the dissolution profiles for the four different active agents formulated as multi-layer tablets. FIG. 5(a) shows comparison of dissolution profiles containing 8403 active 119E (mono-layer tablet), 123E (two-layer tablet) and 127E (three-layer tablet). FIG. 5(b) shows comparison of dissolution profiles containing 8110 active 118E (mono-layer tablet), 122E (two-layer tablet) and 126E (three-layer tablet). FIG. 5(c) shows comparison of dissolution profiles containing 9410 active 121E (mono-layer tablet), 125E (two-layer tablet) and 129E (three-layer tablet). FIG. 5(d) shows comparison of dissolution profiles containing 1022 active 120E (mono-layer tablet), 124E (two-layer tablet) and 128E (three-layer tablet).

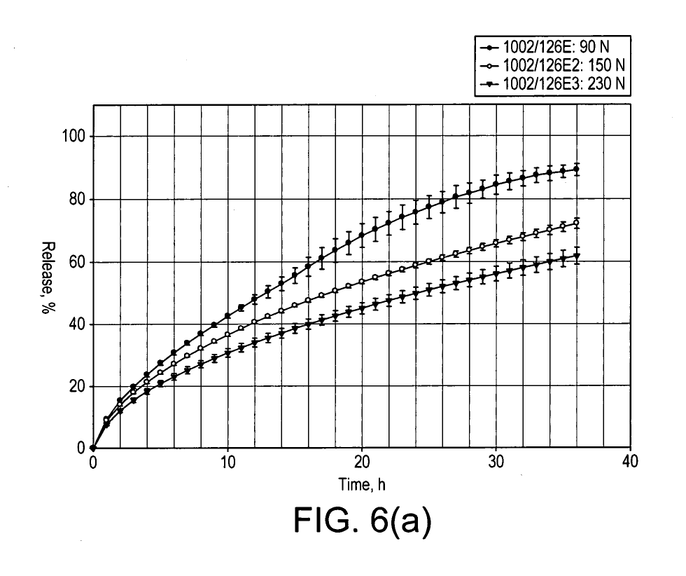

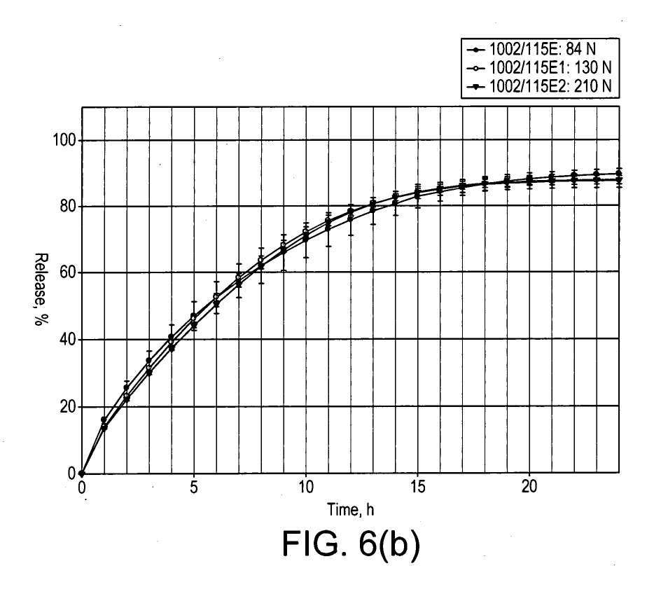

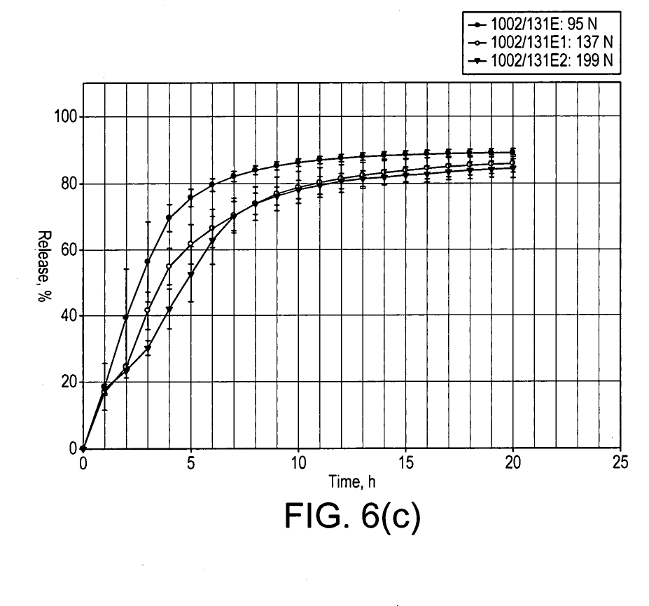

FIG. 6 shows comparison of dissolution profiles for three-layer tablets. FIG. 6(a) shows the dissolution profile for the three-layer tablet containing 8110 active compressed at 89N (126E), 147N (126E2) and 230N (126E3). FIG. 6(b) shows a comparison of dissolution profiles for three-layer tablets containing 8403 active at 84N (115E), 130N (115E1) and 210N (115E2). FIG. 6(c) shows comparison of dissolution profiles for three-layer tablets containing 8403 active and compressed at 95N (131E), at 137N (131E1) and 199N (131E2).

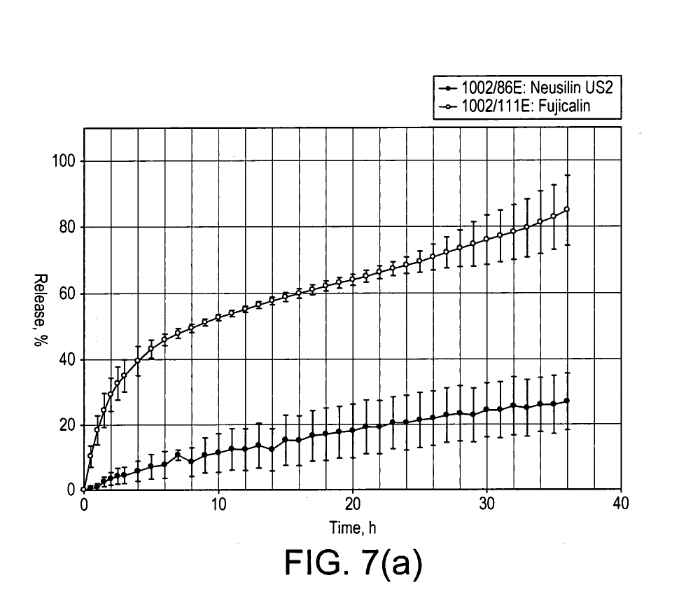

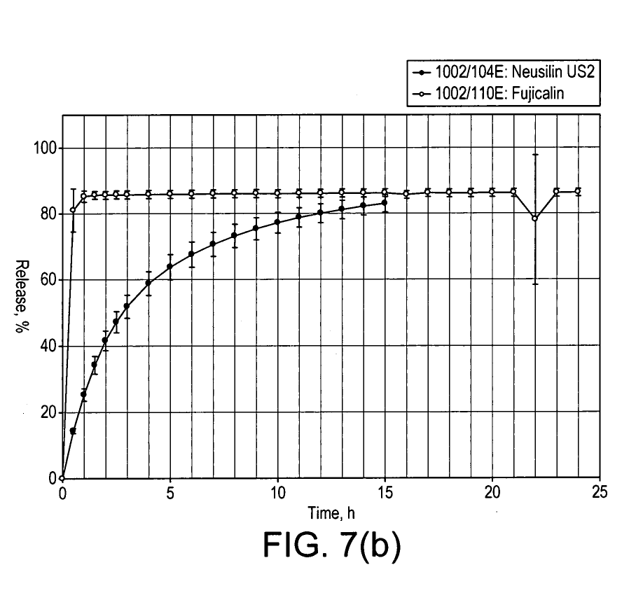

FIG. 7 shows the results of comparative tests with anhydrous dibasic calcium phosphate. FIG. 7(a) shows dissolution profiles of tablets 86E (magnesium aluminometasilicate/Neusilin™) and 111E (calcium phosphate/Fujicalin™). FIG. 7(b) shows dissolution profiles of tablets 104E (magnesium aluminometasilicate/Neusilin™) and 110E (calcium phosphate/Fujicalin™).

### EXAMPLE 1

### Preparation of Tablets Containing Active Formulated in Magnesium Aluminometasilicate

Previous uses of magnesium aluminometasilicate (available as Neusilin™ from Fuji Chemical Co.) have been for flow enhancement, as tablet disintegrant, as stabiliser for deliquescent drugs and for absorption of water or oil. Table 1 shows the characteristics of grades US2 and UFL2 Neusilin™ available from Fuji Chemical Co. USA (http://fujichemusa.com/Neusilin.htm)

TABLE 1

Neusilin ™
Grade

| | US2 | UFL2 |
|---|---|---|
| Form | Spherical | Powder |
| | fine | |
| | granule | |
| Loose bulk density, g/ml | 0.15 | 0.08 |
| Tapped bulk density, g/ml | 0.19 | 0.13 |
| True specific gravity, g/ml | 2.2 | 2.2 |
| Specific surface area, m2/g | 300 | 300 |
| Mean particle size (agglomerate), | 60-120 | 2-8 |
| μm | | |
| Ultimate single mean particle | 20 | 20 |
| size (by SEM), nm | | |
| Angle of repose, ° | 30 | 45 |

| | |
|---|---|
| Composition | Al₂O₃ 29.1-35.5% |
| | MgO 11.4-14% |
| | SiO₂ 29.2-35.6% |
| Solubility | Practically |
| | insoluble |
| | in water |
| | and in ethanol |
| Oil absorbing capacity (ml/g) | 3.2 |

Pharmaceutically active agents can be formulated in magnesium aluminometasilicate as follows. All the excipients described and the active drug were mixed together in a low shear blender (cubic blender) for 15 minutes at 22 rpm until an homogeneous blend is obtained (visually). This blend was then compressed on a single punch press (Korsch EKO) for the monolayer tablets and on a multi-layer rotatory press (Manesty LP 39) for multi-layer tablets.

### EXAMPLE 2

### Controlled Release Properties of Tablet Formulation Including Magnesium Aluminometasilicate

Prototype formulation 86E (3.85% w/w active, 92.3% w/w Neusilin™ US2, 1.44% w/w Aerosil™ 200 and 2.4% w/w Magnesium stearate) was used as reference. New prototypes (88E, 100E, 99E, 89E, 90E and 87E) were prepared where the Neusilin™ content was regularly decreased from 92% w/w to 0% w/w by replacing it by Avicel™ PH102. Dissolution profiles are displayed in FIG. 1.

Profiles reported in FIG. 1 are ranking according to the Neusilin™ content of the tablets. Highest Neusilin™ contents yield slowest release velocities. A high controlled release corresponding to 80% of the active ingredient released in 22 hours is obtained with a tablet containing, at least, 54% w/w Neusilin™ with a filler like Avicel™ PH102.

For all tablets, bubbles emission can be noticed and tablets disintegrate in water more easily when the Avicel™ PH102 content increases. This demonstrates that the porous structure is maintained in the tablet through the compression step in the tabletting procedure.

From these results it can be seen that the controlled release obtained with tablets containing high amounts of Neusilin™ appears to be dependent on the amount of Neusilin™ present in the tablet. At high Neusilin™ contents, a pores network is formed, rugged enough to resist the invasion of water when tablet is immersed. The solubilised active is stressed to follow pores network to reach the dissolution medium. The release is thus slow. At lower Neusilin™ content, the integrity of the network is lost when water invades the pores and the active is released faster.

### EXAMPLE 3

### Influence of Additional Excipients/Adjuvants on Controlled Release Dissolution Profile of Tablet Formulated with Magnesium Aluminometasilicate

Based on same prototype formulation 89E (46.15% w/w Neusilin™ US2, 46.15% w/w Avicel™ PH102, 2.4% w/w Magnesium stearate), tablet 98E is compressed where equal amounts of Neusilin™ and Avicel™ are replaced by Compritol™ 888 ATO (36.5% w/w Neusilin™ US2, 36.5% w/w Avicel™ PH102 and 19.2% w/w Compritol™ 888 ATO). Dissolution profiles of tablets 89E and 98E are compared in FIG. 2.

The addition of Compritol™ 888 ATO in tablet formulation 89E has a big effect on the active ingredient release velocity. For the 98E prototype, 30% of active is released in 9 hours instead of 1 hour for the 89E prototype. The active is, therefore, released faster from tablet 98E (with Compritol™) than from tablet 97E (with Magnesium stearate). The use of waxy or lipid substances such as Compritol™ (glyceryl behenate) reduces the inter porosity of the blend so can therefore reduce the wettability of the tablet and hence reduce the rate of active drug release. Hydrophobic substances such as magnesium stearate also reduce the wettability of the tablet and reduce the rate of active drug release.

### EXAMPLE 4

### Influence of the Ratio of Active/Magnesium Aluminometasilicate

In order to slow down the drug release, the active ingredient/Neusilin™ ratio was increased by adding more Neusilin™ to the reference formulation 104E (this yield heavier tablets).

Reference tablet 104E weighs 160 mg and contains 37.5% w/w active and 60% w/w Neusilin™. Tablets 107E (weight 240 mg, 25% w/w active and 72.5% w/w Neusilin™) and 106E (weight 320 mg, 18.75% w/w active and 78.75% w/w Neusilin™) were prepared by direct compression from blends 30SR and 29SR. Dissolution profiles of tablets 104E, 107E and 106E are displayed in FIG. 3. As can be seen on FIG. 3, decreasing the active ingredient/Neusilin™ ratio allows to slow down the active ingredient release.

### EXAMPLE 5

### Addition of a Barrier Layer

The use of a “barrier” layer or “support platform” to modify the geometry of the tablet in order to increase or to decrease the rate of active drug release from the layer(s) containing the active agent was investigated.

A barrier layer blend D1 was prepared containing 39.875% w/w Methocel™ K100M, 39.875% w/w Lactose, 13.5% w/w Compritol™ 888 ATO, 5% w/w Plasdone™ K29-32, Aerosil™ and Magnesium stearate.

Based on formulation D1, a barrier blend 1002/56B where all the Lactose has been replaced by Neusilin™ US2 was prepared.

Based on formulation D1, a barrier blend 1002/63B was prepared, where all the Lactose had been replaced by Neusilin™ US2 and one half of the Methocel™ K100M had been replaced by Compritol™ 888 ATO and the other half by Avicel™ PH102.

Two layer tablets 108E, 109E and 112E were obtained by compressing 27SR active blend (used for tablet 104E) with support layers D1, 56B and 63B respectively.

Dissolution profiles for tablets 104E, 108E, 109E and 112E are displayed in FIG. 4. Release profiles of two layer tablets are slower than the one of the monolayer tablet 104E. Addition of a barrier reduces contact area between water and active core thus slowing erosion and active ingredient release. Furthermore, one order release profile for the monolayer prototype 104E comes closer to a zero order release profile when a barrier is added.

### EXAMPLE 6

### Multi-Layer Tablets

Four different active agents with different solubilities were selected for the preparation of multi-layer tablets, as follows:

8403 (Diltiazem HCl)—solubility equals to 1200 mg/ml in water.

8110 (Bucindolol)—solubility equals to 257 mg/ml in water.

9410 (Prednisone)—solubility of 0.1 mg/ml in water.

1022—solubility of 0.03 mg/ml in water and 0.14 mg/ml in pH 1.0.

These four actives were formulated as mono-layer tablet with the same dosage strength (10 mg per tablet, 10% w/w) and Neusilin™ US2 (86% w/w).

Very soluble active 8403, freely soluble active 8110, soluble active 9410 and sparingly soluble active 1022 were blended with Neusilin™ US2 to give active blend 1002/39SR, 38SR, 41SR and 40SR respectively.

These blends were then used for the core of the multi-layer tablets. Support layer blend 1002/63B (see above) was used to prepare the support layers.

Active 8403 gave two-layer tablet 1002/123E and three-layer tablet 1002/127E.

Active 8110 gave two-layer tablet 1002/122E and three-layer tablet 1002/126E.

Active 9410 gave two-layer tablet 1002/125E and three-layer tablet 1002/129E.

Active 1022 gave two-layer tablet 1002/124E and three-layer tablet 1002/128E.

These tablets were tested in the same dissolution conditions as mono-layer tablets and results are reported in FIGS. 5(a), 5(b), 5(c) and 5(d).

Whatever the active and its solubility, the addition of one support layer leads to a slowdown of the release. The addition of a second support layer slows down more strongly the active ingredient release.

This effect is also observed and well known with mono-layer tablets of hydroxypropylmethylcellulose polymers when the active ingredient release area is reduced by addition of one or two barrier layers. In the case of Neusilin™ matrix, the decrease of the area available for the active ingredient release could explain the slowing down of the release when barrier layers are added. Addition of barrier layers could also improve the integrity of the tablet (or core).

### EXAMPLE 7

### Effect of Compression on Controlled Release from Tablet Formulated with Magnesium Aluminometasilicate

Ruggedness of active ingredient release toward compression forces applied to the tablet and resulting hardness was investigated.

Bucindolol

Three-layer tablet 1002/126E was compressed till hardness 89 N and compared to the three-layer tablets 1002/126E2 and 1002/126E3 compressed till hardness 147N and 230N respectively.

Dissolution profiles of tablets 126E, 126E2 and 126E3 are displayed in FIG. 6(a). As can be seen in FIG. 6(a), the final hardness of the three-layer tablet has a big impact on the active ingredient release rate. The more compressed tablet gives the slower release. Such influence is not so pronounced on HPMC multi-layer tablets. In the case of Neusilin™ tablet, the effect of compression forces could be due to the fact that the integrity of the tablet is improved when it is compressed harder and that porosity is reduced (as the dimension of the tablet decreases) when the tablet is compressed harder.

These findings show that Neusilin™ systems are sensitive to tablet hardness and compression forces which make them not too rugged. At the same time this parameter should allow the fine tuning of the active ingredient release rate.

Diltiazem HCl

A reference prototype formulation Diltiazem HCl composed of a trilayer tablet consisting of a 33SR active layer in between two support layers L1 was modified as follows.

33SR active blend contains mainly 46.875% w/w active, 36.5% w/w Methocel™ K100M and 10.4% w/w Mannitol 60™. L1 support layer contains mainly 80.39% w/w Methocel™ K100M.

Based on L1 formulation, support layer 1002/64B was prepared where half of the Methocel™ K100M has been replaced by Neusilin™ US2. Based on 33SR formulation, active blend 35SR was prepared where one third of the Methocel™ K100M and all Mannitol 60™ have been replaced by Neusilin™ US2 (Neusilin™ content equals 30% w/w).

Active blend 35SR was compressed with support layers 64B to give tri-layer tablet 115E (35SR+2x64B). Prototype 1002/115E (Diltiazem HCl with Neusilin™) was thus prepared with hardness 130N (1002/115E1) and 210N (1002/115E2).

Dissolution profiles of tablets 115E, 115E1 and 115E2 are displayed in FIG. 6(b).

On the three-layer prototype 115E, major changes in tablet hardness do not lead significantly different active ingredient release rates. This could be explained by the fact that, in this case, core and barriers formulations consist of a blend of Methocel™ and Neusilin™ and not in pure Neusilin™. The active ingredient release is thus due to the HPMC network (which swells and gels) and to the Neusilin™ network.

A new Diltiazem HCl matrix was prepared where the whole quantity of Methocel™ has been replaced by Neusilin™. Resulting active blend 42SR was compressed with two 64B support layers to give prototype 131E (95N), 131E1 (137N) and 131E2 (199N).

### COMPARATIVE EXAMPLE 1

### Formulation of Active with Anhydrous Dibasic Calcium Phosphate (Fujicalin™)

Fujicalin™ is anhydrous dibasic calcium phosphate available from Fuji Chemical Co. (http://fujichemusa.com/fujicalin.htm). Chemically it is the same as conventional products (Emcompress™) but Fujicalin™'s high porosity and large specific surface area creates totally different characteristics. Unique features of Fujicalin™ are its large specific surface area, its high absorption capacity and its high compressibility. It has been used previously for flow enhancement, as tablet disintegrant and for absorption of water or oil. Table 2 shows the characteristic features of Fujicalin™.

TABLE 2

| | Grade |
|---|---|
| | Fujicalin ™ |
| Loose bulk density, g/ml | 2.5 |
| Tapped bulk density, g/ml | 2.2 |
| Specific surface area, m2/g | 40 |
| Mean particle size (agglomerate), μm | 115 |
| Angle of repose, ° | 32 |
| Water absorbing capacity (ml/g) | 0.98 |

Fujicalin™'s properties described by the manufacturer are therefore quite similar to the properties claimed for Neusilin™. It was decided to investigate whether this material with described properties and uses similar to Neuslin™ exhibited any controlled release properties in a tablet formulation.

Active 1022

Based on blend and tablet formulation 16SR and 86E containing 92.3% w/w Neusilin™ and 3.85% w/w active 1022, blend 32SR and corresponding tablet 111E were prepared where Neusilin™ has been replaced by Fujicalin™. Dissolution profiles of tablets 86E and 111E are compared in FIG. 7(a).

Active ingredient release obtained with Fujicalin™ is far faster than the one obtained with Neusilin™. Tablet disintegration with Fujicalin™ is far faster as well.

Active 8403

Based on blend and tablet formulation 27SR and 104E containing 60% w/w Neusilin™ and 37.5% w/w active 8403, blend 31SR and corresponding tablet 110E were prepared where Neusilin™ has been replaced by Fujicalin™. Dissolution profiles of tablets 104E and 110E are compared in FIG. 7(b).

With this active, the Fujicalin™ matrix leads to a faster active ingredient release than with the Neusilin™ matrix.

In both cases (active 1022 and 8403) matrix obtained with Fujicalin™ is less robust than the Neusilin™ matrix.

Conclusion

From these results, it can therefore be concluded that materials that are known to act as absorbents are not inherently able to act as controlled release excipients for formulation of active agents in tablets for oral administration.

1. A dosage form for oral administration consisting of a minimum 15% w/w of magnesium aluminometasilicate, one or more pharmaceutically active agents and optionally one or more pharmaceutically acceptable diluents.

2. The dosage form of claim 1, in which the magnesium aluminometasilicate is present in the range of from 15% to 95%

3. The dosage form as claimed in claim 1, in which the pharmaceutically active agent present is a drug substance.

4. The dosage form of claim 1, in which the pharmaceutically active agent present is a supplement.

5. The dosage form of claim 1, in which the active substance is contained in a percentage between 0.05% to 50% by weight of the dosage form

6. The dosage form of claim 1, in which the dosage form is a multi-layer tablet comprising one or more layers containing an active agent.

7. The dosage form of claim 1, in which the dosage form is a compression coated tablet

8. A dosage form for oral administration consisting of a minimum 15% w/w of magnesium aluminometasilicate, a pharmaceutically active agent, a pharmaceutically acceptable lipid excipient and optionally one or more pharmaceutically acceptable diluents.

9. The dosage form of claim 8, in which the lipid excipient is a lipoid or waxy compound.

10. The dosage form of claim 8, in which the lipid excipient is microcrystalline or glyceryl behenate.

11. A method for controlling the release of a pharmaceutically active agent from a dosage form, the method comprising the step of formulating the active agent in a granulate composition comprising a minimum 15% w/w magnesium aluminometasilicate.

12. The use of magnesium aluminometasilicate as a controlled-release excipient in the formulation of a pharmaceutically active substance in a dosage form.
